(19) United States
(12) Patent Application Publication
(10) Pub. No.: US 2021/0386782 A1
(43) Pub. Date: Dec. 16, 2021

(54) Near Infrared Photoimmunotherapy

(71) Applicant: The Brigham and Women`s Hospital, Inc., Boston, MA (US)

(72) Inventor: Rachael A. Clark, Belmont, MA (US)

(21) Appl. No.: 17/286,194

(22) PCT Filed: Oct. 18, 2019
(86) PCT No.: PCT/US19/56897

§ 371 Date: Apr. 16, 2021

Related U.S. Application Data
(60) Provisional application No. 62/747,526, filed on Oct. 18, 2018.

(51) Int. Cl. A61K 35/17; C07K 16/28; A61K 41/00; A61K 47/68

(52) U.S. Cl. CPC A61K 35/17; A61K 47/6849; A61K 41/0071; C07K 16/2809

(57) ABSTRACT

Compositions comprising memory T cell Targeting Constructs comprising an anti-T cell antibody or antigen-binding portion thereof linked to a near-infrared photoactivated cytotoxin, e.g., a photoactive adduct of an infrared dye, and methods of use thereof for reducing numbers of pathogenic T cells selectively in peripheral tissues e.g., for treating inflammatory and autoimmune diseases.

### CLAIM OF PRIORITY

This application claims the benefit of U.S. Provisional Patent Application Ser. No. 62/747,526, filed on Oct. 18, 2018. The entire contents of the foregoing are hereby incorporated by reference.

### FEDERALLY SPONSORED RESEARCH OR DEVELOPMENT

This invention was made with Government support under Grant No. AR063962 awarded by the National Institutes of Health. The Government has certain rights in the invention.

### TECHNICAL FIELD

Described herein are compositions comprising memory T cell Targeting Constructs comprising an anti-T cell antibody or antigen-binding portion thereof linked to a near-infrared photoactivated cytotoxin, e.g., a photoactive adduct of an infrared dye, and methods of use thereof for reducing numbers of TRM in a tissue, e.g., for treating inflammatory and autoimmune diseases.

### BACKGROUND

Resident memory T cells (TRM) are nonrecirculating T cells that remain long-term in peripheral tissues such as the lung, gut and skin. Memory T cells are T cells that have undergone the naïve to memory transition. Pathogenic T cells are T cells that, by their activation, cytokine production or other effector functions, contribute to inappropriate inflammation.

### SUMMARY

Provided herein are Memory T cell targeting constructs comprising an anti-T cell antibody or antigen-binding portion thereof that binds to an antigen present on the surface of a memory T cell, linked to a near-infrared photoactivated cytotoxin.

In some embodiments, the anti-T cell antibody or antigen-binding portion thereof that binds to CD3, CD4, CD8, CD103, C-X-C motif chemokine receptor 6 (CXCR6), or CD69.

In some embodiments, the near-infrared photoactivated cytotoxin comprises a phthalocyanine dye. In some embodiments, the phthalocyanine dye is IRDYE 700DX.

In some embodiments, the near-infrared photoactivated cytotoxin comprises a light-activated Platinum IV (PtIV) complex or photoactivatable Re(I) complex and lanthanide-doped upconversion nanoparticles (UCNPs). In some embodiments, the light-activated PtIV complex comprises trans, trans, trans-[Pt(N3)₂(OH)2(NH3)(py)].

In some embodiments, the antibody is a deimmunised, humanized or fully human antibody.

Also provided herein are methods of reducing inflammation in a tissue in a subject. The methods include (i) administering to the subject an effective amount of a T cell Targeting Construct as described herein, and (ii) administering to the tissue a near infrared (NIR) light sufficient to activate the cytotoxin. Also provided is the T cell Targeting Constructs as described herein for use in a method of reducing inflammation in a tissue in a subject.

In some embodiments, the NIR light is a wavelength of 660 to 740 nm.

In some embodiments, the T cell Targeting Construct is administered cutaneously or parenterally. In some embodiments, the parenteral administration is intravenous, intraperitoneal, subcutaneous, or intramuscular.

In some embodiments, the subject has an inflammatory or autoimmune condition/disease.

In some embodiments, the subject has skin lesions associated with psoriasis, atopic dermatitis, lupus, vitiligo, graft-versus-host disease, cutaneous T-cell lymphoma, contact dermatitis, cutaneous hypersensitivity response, lichen planus, lichen planopilaris, rejection of vascularized composite allografts, alopecia areata, scarring alopecia or sarcoid and the methods include administering the NIR light to the skin lesions.

In some embodiments, the subject has T cell mediated kidney or renal pathology/disease associated with lupus nephritis, autoimmune nephritis or kidney graft rejection and the methods include administering the NIR light to the kidney.

In some embodiments, the subject has gastrointestinal inflammation as a result of an autoimmune and/or an inflammatory condition, e.g., inflammatory bowel disease (Crohn's disease, ulcerative colitis), and the methods include administering the NIR light to the gut or bowel of the subject.

In some embodiments, the subject has joint inflammation from rheumatoid arthritis or spondyloarthritides, and the methods include administering the NIR light to affected joints.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a schematic illustration of selective targeting of pathogenic T cells in tissues by NIR-active anti-T cell conjugates. Systemic or local administration of a T cell targeting construct as described herein plus local administration of near infrared (NIR) light to treat inflammatory diseases of the skin (e.g. psoriasis, cutaneous T cell lymphomas, vitiligo, atopic dermatitis, graft vs. host disease, contact dermatitis), the joints (rheumatoid arthritis, spondyloarthropathies), the lungs (asthma), the kidneys (e.g., nephritis, graft rejection) or to the gastrointestinal track via endoscope (e.g. Crohn's disease, ulcerative colititis) to safely deplete inflammatory T cells specifically within affected tissues, leading to disease remission without systemic immunosuppression.

FIGS. 2A-2C are graphs showing evidence that IR700-conjugated antibodies+NIR light deplete T cells and specific T cell subsets in vitro. T cells were isolated from peripheral blood of healthy human donors by Ficoll centrifugation. FIG. 2A, Treatment of cells in vitro with both anti-CD3-IR700 and near infrared light (αCD3+NIR) led to near total depletion of CD3+ T cells. However, treatment with anti-CD3-IR700 (αCD3) or near infrared light (NIR) alone, or with NIR and an IR700 conjugated isotype control antibody, did not deplete T cells. FIG. 2B, CD4+ T cells were selectively depleted by anti-CD4-IR700 plus near infrared light (αCD4+NIR) but CD8+ T cells, which lack CD4, were spared. FIG. 2C, CD8+ T cells were selectively depleted by anti-CD8-IR700 plus near infrared light (αCD8+NIR).

FIGS. 3A-3B are schematic illustrations of the in vivo model used to confirm the ability of the anti-T cell antibodies+NIR to selectively deplete human T cells in skin. FIG. 2A, The model: NSG mice grafted with adult human skin and subsequently infused IV with PBMC from a second human donor develop a GvHD-like dermatitis within the human skin graft (Watanabe, R. et al. (2015). Science Translational Medicine 7(279): 279ra239). FIG. 2B, Pilot data was generated by infusing mice with established GVHD-like dermatitis IV with an anti-CD3 antibody conjugated to IR700 and then exposing the skin graft to near-infrared light. T cells were then isolated from the skin graft and spleen and 24 hours later and analyzed by flow cytometry.

FIGS. 4A-4F are graphs showing that activated and resident memory T cells are effectively depleted from skin by photoimmunotherapy. FIG. 4A, Skin grafts isolated from the skin graft of human engrafted mice contain both activated and resident memory T cells that express CD69 (expressed by both activated and resident memory T cells) and CD103 (expressed by resident memory T cells). T cells isolated from the skin of treated human engrafted mice (as described in FIG. 3A) are shown. FIG. 4B, Activated and resident T cells are effectively depleted from skin by photoimmunotherapy. Histograms of T cells isolated from the skin grafts of human engrafted mice treated with Anti-CD3-IR700 alone (left panels) vs. Anti-CD3-IR700 with near-infrared treatment of the skin graft (right panels) are shown. CD4+, CD8+, activated (CD69+) and resident memory (CD103) T cells are all efficiently depleted from skin in the presence of both the Anti-CD3-IR700 and NIR, but not by Anti-CD3-IR700 alone. This confirms that NIR light exposure allows selective depletion of T cells only in the NIR treated skin. FIG. 4C, Bar graphs show the reduction in absolute number of the indicated T cell subsets after treatment with Anti-CD3-IR700 alone (black bars) vs. Anti-CD3-IR700 plus NIR to the human skin graft (white bars). FIG. 4D, shows the % reduction in the indicated T cell subsets in skin after treatment with Anti-CD3-IR700 plus NIR. FIG. 4E, Anti-CD3-IR700+NIR does not deplete T cells in the spleen. The bar graph shows the absolute number of T cells expressing the indicated marker in the spleen after treatment with Anti-CD3-IR700 alone (black bars) vs. Anti-CD3-IR700 plus NIR to the human skin graft (white bars). This data shows that T cells in the circulation are not depleted by this therapy.

FIG. 5 is a set of graphs showing selective depletion of T cells in NIR treated skin in vivo using a second T cell targeting antibody. Shown are the absolute number of T cells in blood, spleen and skin following treatment with anti-CD8-IR700 alone (αCD8) or with a combination of anti-CD8-IR700 and near infrared light delivered to the human skin graft (αCD8+NIR). This data shows that CD8 conjugated to IR700 effectively and selectively depleted CD8 T cells in skin but not in the blood or spleen.

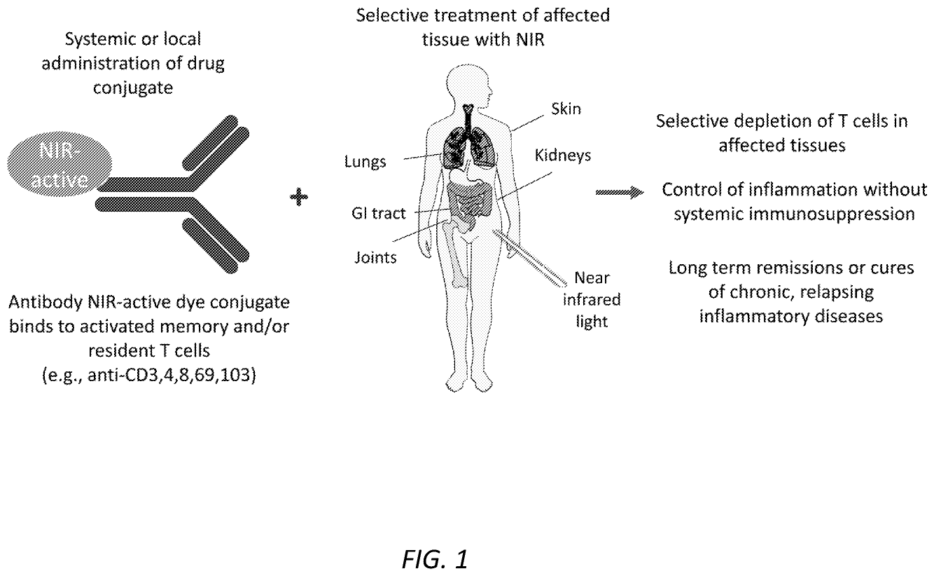

### DETAILED DESCRIPTION

Pathogenic Activated Memory T Cells Cause Chronic Inflammatory Diseases

Inappropriately activated and/or autoreactive (pathogenic) memory T cells cause a variety of relapsing, remitting autoimmune and inflammatory disorders in peripheral tissues, including but not limited to psoriasis, atopic dermatitis, asthma, lupus erythematosus, rheumatoid arthritis and inflammatory bowel disease (Bluestone, J. A., et al., J Clin Invest 125(6): 2250-2260). Patients with severe inflammatory and autoimmune disorders are currently treated with systemically administered immunosuppressants, including but not limited to cyclosporine, mycophenolate mofetil, and an array of anti-inflammatory biologic medications, all of which increase the risk of serious and potentially fatal infections. This invention describes a method by which NIR active antibodies targeting T cells can be combined with exposure of the affected tissue sites to NIR, in order to effectively and selectively deplete T cells only within the affected tissues. This approach will control T cell mediated inflammation in the affected tissue sites but would spare T cells in the rest of the body, thereby greatly reducing or eliminating the risk of infection.

Pathogenic Resident Memory T Cells Drive Recurrence of Chronic Inflammatory Diseases

It is now recognized that a growing number of chronic, relapsing inflammatory diseases are caused by a specific subtype of memory T cells, termed resident memory T cells (TRM). TRM are non-recirculating memory T cells that persist long term in peripheral tissues. TRM persist in the absence of antigen, have strong effector functions and provide rapid on-site immune protection against known pathogens in peripheral tissues. TRM provide rapid immune protection against pathogens but autoreactive, aberrantly activated and malignant resident memory cells contribute to numerous human inflammatory diseases. TRM are responsible for the recurrent inflammatory lesions in mycosis fungoides, psoriasis, vitiligo, and likely many other inflammatory diseases, including but not limited to rheumatoid arthritis, inflammatory bowel disease, atopic dermatitis and lupus nephritis (Clark, R. A. (2015). Sci Transl Med 7(269): 269rv261; Richmond, J. M et. al.. (2018) Sci Transl Med 10(450)). Once pathogenic TRM develop within affected tissues, they are long lived and very difficult to kill. Current therapeutic approaches, including corticosteroids, immunosuppressants and biologic anti-cytokine medications, suppress activation but do not kill these cells. As a result, there is often relapse of inflammation after the withdrawal of medication. If an effective method could be developed to kill these pathogenic T cells selectively within the affected peripheral tissues, it could lead to long-term remissions or cures in TRM mediated diseases without the infectious risks that accompany systemic depletion of T cells and the use of systemic immunosuppressants. This invention uses NIR active antibodies targeting T cells antigens, combined with exposure of the affected tissue sites to NIR, in order to effectively and selectively deplete T cells—including the seed population of resident T cells that drive the disease.

The present methods use an antibody-photoactive molecule conjugate to reduce numbers of pathogenic activated and/or resident T cells in the peripheral tissues of subjects with autoimmune and inflammatory disorders. In these conjugates, antibodies that bind to antigens present on the surface of human T cells (e.g., CD3, CD4, CD8, CD103, C-X-C motif chemokine receptor 6 (CXCR6), and CD69), referred to herein as anti-T cell antibodies, are linked covalently to a photoactive adduct IRDye, e.g., 700DX, and the conjugate is activated by near infrared (NIR) light to kill cells expressing the antigen. Once injected, anti-T Cell-IR700 binds to the appropriate cell surface antigen (e.g., CD3, CD4, CD8, CD103, C-X-C motif chemokine receptor 6 (CXCR6), and CD69), and the photoactivatable dye, e.g., silicaphthalocyanine dye (IRDye 700DX), kills cells by inducing membrane damage after NIR light exposure. The NIR light exposure (e.g., 690 nm) induces highly selective cell death within minutes, without damaging nearby cells. In some embodiments, the antibody conjugates are injected intravenously, and then the affected areas of the body (e.g., psoriatic skin lesions in psoriasis, the back overlying the kidneys in lupus nephritis, the areas overlying joints in rheumatoid arthritis) are treated with NIR. NIR is harmless in itself and capable of penetrating deeply into the body. For example, NIR alone has shown promise in the treatment of traumatic brain injury (Henderson, T. A. (2016) Neural Regen Res 11(4): 563-565).

T Cell Targeting Construct

The present methods and compositions include T Cell Targeting Constructs comprising an anti-T cell antibody or antigen-binding portion thereof linked to a near-infrared photoactivated cytotoxin, e.g., a photoactive adduct of an infrared dye.

Anti-T Cell Antibody

The term “antibody” as used herein refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments the antibody has effector function and can fix complement. In some embodiments, the antibody has reduced or no ability to bind an Fc receptor. For example, the antibody can be an isotype or subtype, fragment or other mutant, which does not support binding to an Fc receptor, e.g., it has a mutagenized or deleted Fc receptor binding region. Methods for making antibodies and fragments thereof are known in the art, see, e.g., Harlow et. al., editors, Antibodies: A Laboratory Manual (1988); Goding, Monoclonal Antibodies: Principles and Practice, (N.Y. Academic Press 1983); Howard and Kaser, Making and Using Antibodies: A Practical Handbook (CRC Press; 1st edition, Dec. 13, 2006); Kontermann and Dübel, Antibody Engineering Volume 1 (Springer Protocols) (Springer; 2nd ed., May 21, 2010); Lo, Antibody Engineering: Methods and Protocols (Methods in Molecular Biology) (Humana Press; Nov. 10, 2010); and Dübel, Handbook of Therapeutic Antibodies: Technologies, Emerging Developments and Approved Therapeutics, (Wiley-VCH; 1 edition Sep. 7, 2010). Useful anti-TRM antibodies include those that bind to CD3, CD4, CD8, CD103, C-X-C motif chemokine receptor 6 (CXCR6), and CD69.

CD3

CD3 is expressed by all T cells and targeting this antigen has the potential to deplete all T cells from an affected tissue. Accession numbers for exemplary sequences for human CD3 proteins are shown in the following table.

| | GenBank |
|---|---|
| CD3 isoform | RefSeq ID |
| T-cell surface glycoprotein CD3 delta chain isoform A | P_000723.1 |
| precursor | |
| T-cell surface glycoprotein CD3 delta chain isoform B | NP_001035741.1 |
| precursor | |
| T-cell surface glycoprotein CD3 epsilon chain | P07766.2 |
| precursor | |
| T-cell surface glycoprotein CD3 gamma chain | NP_000064.1 |
| precursor | |
| T-cell surface glycoprotein CD3 zeta chain isoform 1 | NP_932170.1 |
| precursor | |
| T-cell surface glycoprotein CD3 zeta chain isoform 2 | NP_000725.1 |
| precursor | |

Anti-CD3 antibodies include, but are not limited to, those disclosed in US20150166661, US20170204194, U.S. Pat. Nos. 7,728,114, 8,551,478, US20140193399, US20030216551, US20060275292, WO2017136659, and WO2016179003. Anti-CD3 antibodies specific for human CD3 available from commercial suppliers, include, but are limited to, clone 289-13801 (Cat. No. ABIN234581, Antibodies-Online), clone 4AOKT3 (Cat. No. ABIN2145039, Antibodies-Online), clone 4D10A6 (Cat. No. ABIN969472), clone B477 (Cat. No. ABIN965782, Antibodies-Online), clone B-B11 (Cat. No. ABIN1383795, Antibodies-Online), clone hCD3 (Cat. No. ABIN2136389, Antibodies-Online), clone HIT3a (Cat. No. ABIN2136387, Antibodies-Online), clone Okt 03 (Cat. No. ABIN457398, Antibodies-Online), clone UCHT1 (Cat. No. ABIN135720, Antibodies-Online), clone BC3 (Cat. No. 830301, BioLegend), clone Hu113 (Cat. No. MAB9929-100, R&D Systems Inc.), clone B-B11 (Cat. No. AM31215PU-N, Origene), clone N26-R (Cat. No. NBP1-79054, Novus Biologicals Canada), clone 1A7E5G5 (Cat. No. 10977-MM03, Sino Biological Inc), clone UCHT-1 (Cat. No. T-1363, BMA Biomedicals).

CD4

CD4 is expressed by helper T cells. Targeting this antigen could be used to selectively deplete CD4 T cells in diseases where CD4 T cells preferentially contribute to pathology. For example, malignant T cells in cutaneous T cell lymphoma are usually CD4+ and targeting these cells could be used to selectively deplete malignant T cells from skin without harming the CD8+ T cell population.

The sequence for human CD4 protein is available in GenBank at Acc. No. NP_000607.1. Anti-CD4 antibodies include, but are not limited to, those disclosed in U.S. Pat. Nos. 7,452,534, 5,871,732, 8,877,913, 8,399,621, 7,947,272, 7,452,981, 8,440,806, 8,586,715, 8,673,304, and 8,685,651. Anti-CD4 antibodies specific for human CD4 available from commercial suppliers, include, but are not limited to, clone 8 (Cat. No. 10400-MM08, Sino Biological Inc.), clone 22 (Cat. No. 10400-MM22, Sino Biological Inc.), clone 6F7B4C5 (Cat. No. 10400-MM03, Sino Biological Inc.), clone CE9.1 (Cat. No. A1091-200, Biovision Inc.), clone CL0395 (Cat. No. AMAb90754, Atlas Antibodies), clone 34915 (Cat. No. MAB3791, R&D Systems), clone 34930 (Cat. No. MAB379-100, R&D Systems), clone 10B5 (Cat. No. GTX84720, GeneTex), clone 13B8.2 (Cat. No. GTX44212, GeneTex), clone MEM-241 (Cat. No. GTX21089, GeneTex), clone 4A11 (Cat. No. ABIN2136522, Antibodies Online), clone 4B12 (Cat. No. ABIN180655, Antibodies Online), and clone 6 Eli) (Cat No. ABIN2136524, Antibodies Online).

CD8 CD8 is expressed by cytotoxic T cells. In some inflammatory diseases, such as allograft rejection, CD8+ T cells are thought to cause the majority of tissue damage (Harper, S. J. et al., (2015). Proc Natl Acad Sci USA 112(41): 12788-12793). Thus, depending on the biology of the inflammatory process, it may be desirable to deplete CD8+ T cells without harming other T cell subsets. The sequence for human CD8 protein is available in GenBank at Acc. No. NP_001759.3. Anti-CD8 antibodies include, but are not limited to, those disclosed in U.S. Pat. No. 9,518,131, WO9015152, and US20090304659. Anti-CD8 antibodies specific for human CD8 available from commercial suppliers, include, but are not limited to clone C8/144B (Cat. No. 925-MSM2-P1, Enquire Bioreagents), clone C8/468 (Cat. No. 925-MSM1-P1, Enquire Bioreagents), clone 37006 (Cat. No. MAB1509, R&D Systems), clone 2ST8.5H7 (Cat. No. GTX75282, GeneTex), clone LT8 (Cat. No. LT8, GeneTex), clone OKT-8 (Cat. No. GTX14198, GeneTex), clone Bu88 (Cat. No. AM05583PU-N, Origene Technologies), clone B-Z31 (Cat. No. AM31251PU-N, Origene Technologies), clone MCD8 (Cat. No. AM39011PU-N, Origene Technologies), clone RAVB3 (Cat. No. AM06078PU-N, Origene Technologies), clone RFT-8 (Cat. No. AM08158PU-N, Origene Technologies), clone 14 (Cat. No. NBP2-50467, Novus Biologicals Canada), clone X107 (Cat. No. NBP2-50469, Novus Biologicals Canada), and clone UCH-T4 (Cat. No. NBP2-50468, Novus Biologicals Canada).

CD103

CD103 is expressed by TRM in peripheral tissues in both humans and mice and is enriched on TRM that populate mucosae and epithelia (Sathaliyawala, T., et al., (2013). Immunity 38(1): 187-197). CD103 is also known as integrin subunit alpha E (ITGAE). The sequence for human CD103 protein is available in GenBank at Acc. No. NP_002199.3. Anti-CD103 antibodies include, but are limited to, those disclosed in US20110142861, US20110142860, and US20050266001. Anti-CD103 antibodies specific for human CD103 available from commercial suppliers, include, but are limited to, clone B-Ly7 (Cat. No. NBP1-43370H, Novus Biologicals Canada), clone BP6 (Cat. No. NBP2-50446H, Novus Biologicals Canada), clone LF61 (Cat. No. NB100-65272H, Novus Biologicals Canada), clone AX.14 (Cat. No. AM05205PU-N, Origene Technologies), clone B-ly7 (Cat. No. AM39027PU-N, Origene Technologies), clone 3H1798 (Cat. No. C2445-63A, United States Biological), clone 3H1797 (Cat. No. C2445-63, United States Biological), clone 3H1797 (Cat. No. C2445-63J1, United States Biological), and clone 3H1797 (Cat. No. C2445-63K, United States Biological).

CXCR6

CXCR6 is expressed by TRM in tissues and is required for their optimal development (Zaid, A., (2017). J Immunol 199(7): 2451-2459). The sequence for human CXCR6 protein is available in GenBank at Acc. No. NP_006555.1. Anti-CXCR6 antibodies include, but are limited to, those disclosed in U.S. Pat. No. 9,872,905 and WO2004019046. Anti-CXCR6 antibodies specific for human CXCR6 available from commercial suppliers, include, but are limited to, clone 56811 (Cat. No. MAB699-100, R&D Systems), clone MM0226-2B44 (Cat. No. NBP2-12243, R&D Systems), clone 14L333 (Cat. No. 216429, R&D Systems), clone K041E5 (Cat. No. 356001, BioLegend), clone K041E5 (Cat. No. 356002, BioLegend), and select polyclonal antibodies specific for human CXCR6 (e.g., Cat. No. GTX77935, GeneTex; Cat. No. SP1286P, Origene Technologies; Cat. No. NLS1102, Novus Biologicals Canada; Cat. No. abx148716, Abbexa; Cat. No. 170358, United States Biological).

CD69

CD69 is a surface molecule that is expressed at high and constant levels by TRM regardless of activation status in all tissues tested so far, and is the most inclusive marker of TRM in human skin (Watanabe, R. et al. (2015). Science Translational Medicine 7(279): 279ra239). CD69 is also expressed by activated T cells in tissues, e.g., at inflamed sites, and is upregulated in vitro within 12 hours of stimulation. CD69 is not expressed by circulating T cells or FOXP3 regulatory T cells, at least in human skin (Clark, R. A., et al. (2007). Blood 109(1): 194-202).

The sequence for human CD69 protein is available in GenBank at Acc. No. NP_001772.1. Anti-CD69 antibodies known in the art and useful in the present methods include, but are not limited to, those disclosed in US20150118237, U.S. Pat. No. 8,440,195, US20130224111, U.S. Pat. Nos. 7,867,475, 8,182,816, WO2018074610, and WO2018150066. Anti-CD69 antibodies specific for human CD19 are available from commercial suppliers, include, but are limited to, clone 4AF50 (Cat. No. ABIN2145225, Antibodies-Online), clone FN50 (Cat. No. ABIN302090, Antibodies-Online), clone 298633 (Cat. No. MAB2359-SP, R&D Systems), clone 298614 (Cat. No. MAB23591, R&D Systems), monoclonal anti-CD69 antibody (Cat. No. AM03132PU-N, OriGene TEchnologies), clone 15B5G2 (Cat. No. NBP2-25242SS, Novus Biologicals Canada), clone 7H192 (Cat. No. C2424-01E, US Biological Life Sciences), clone 4H3 (Cat. No. 124672, US Biological Life Sciences), clone 7H192 (Cat. No. C2424-01, US Biological Life Sciences), clone HP-4B3 (Cat. No. LS-C134543-100, LifeSpan BioSciences), or select polyclonal antibodies specific for human CD69 (e.g., Cat. No. ABIN2136942, Antibodies-Online; Cat. No. AF2359, R&D Systems; Cat. No. GTX37447, GeneTex Inc.; Cat. No. AP21168PU-N, OriGene Technologies; Cat. No. 124671, US Biological Life Sciences).

Methods for humanizing any of these antibodies, or for generating fully human antibodies, are known in the art. Typically, “humanization” results in an antibody that is less immunogenic, with complete retention of the antigen-binding properties of the original molecule. In order to retain all the antigen-binding properties of the original antibody, the structure of its combining-site has to be faithfully reproduced in the “humanized” version. This can potentially be achieved by transplanting the combining site of the nonhuman antibody onto a human framework, either (a) by grafting the entire nonhuman variable domains onto human constant regions to generate a chimeric antibody (Morrison et al., Proc. Natl. Acad. Sci., USA 81:6801 (1984); Morrison and Oi, Adv. Immunol. 44:65 (1988) (which preserves the ligand-binding properties, but which also retains the immunogenicity of the nonhuman variable domains); (b) by grafting only the nonhuman CDRs onto human framework and constant regions with or without retention of critical framework residues (Jones et al. Nature, 321:522 (1986); Verhoeyen et al., Science 239:1539 (1988)); or (c) by transplanting the entire nonhuman variable domains (to preserve ligand-binding properties) but also “cloaking” them with a human-like surface through judicious replacement of exposed residues (to reduce antigenicity) (Padlan, Molec. Immunol. 28:489 (1991)).

Humanization by CDR grafting typically involves transplanting only the CDRs onto human fragment onto human framework and constant regions. Theoretically, this should substantially eliminate immunogenicity (except if allotypic or idiotypic differences exist). However, it has been reported that some framework residues of the original antibody also need to be preserved (Riechmann et al., Nature 332:323 (1988); Queen et al., Proc. Natl. Acad. Sci. USA 86:10,029 (1989)). The framework residues which need to be preserved can be identified by computer modeling. Alternatively, critical framework residues may potentially be identified by comparing known antibody combining site structures (Padlan, Molec. Immun. 31(3):169-217 (1994)). The invention also includes partially humanized antibodies, in which the 6 CDRs of the heavy and light chains and a limited number of structural amino acids of the murine monoclonal antibody are grafted by recombinant technology to the CDR-depleted human IgG scaffold (Jones et al., Nature 321:522-525 (1986)).

Deimmunized antibodies are made by replacing immunogenic epitopes in the murine variable domains with benign amino acid sequences, resulting in a deimmunized variable domain. The deimmunized variable domains are linked genetically to human IgG constant domains to yield a deimmunized antibody (Biovation, Aberdeen, Scotland).

The antibody can also be a single chain antibody. A single-chain antibody (scFV) can be engineered (see, for example, Colcher et al., Ann. N. Y. Acad. Sci. 880:263-80 (1999); and Reiter, Clin. Cancer Res. 2:245-52 (1996)). The single chain antibody can be dimerized or multimerized to generate multivalent antibodies having specificities for different epitopes of the same target protein. In some embodiments, the antibody is monovalent, e.g., as described in Abbs et al., Ther. Immunol. 1(6):325-31 (1994), incorporated herein by reference.

Near-Infrared Photoactivated Cytotoxin

The T cell targeting constructs include a NIR-photoactivated cytotoxin. A number of such cytotoxins are known in the art, including phthalocyanine dyes such as IRDYE 700DX, which exerts its cytotoxic effect through photoactivation with a near-infrared laser. Alternatively, light-activated PtIV complexes, e.g., trans, trans, trans-[Pt(N₃)₂(OH)₂(NH₃)(py)] (Mackay et al., PNAS 104(52):20743-20748 (2007)); photoactivatable Re(I) complexes and lanthanide-doped upconversion nanoparticles (UCNPs) (Hu et al., Dalton Trans., 2016, 45, 14101-14108).

The NIR-photoactivated cytotoxin, e.g., the IRDYE 700DX, may have an NHS ester linkage to allow for conjugation to the antibody. For example, the a primary amine (e.g., an amino group) in the antibody can be used to link to the NHS ester of 700DX via an amide bond. The NHS ester IRDYE 700DX has the following structure:

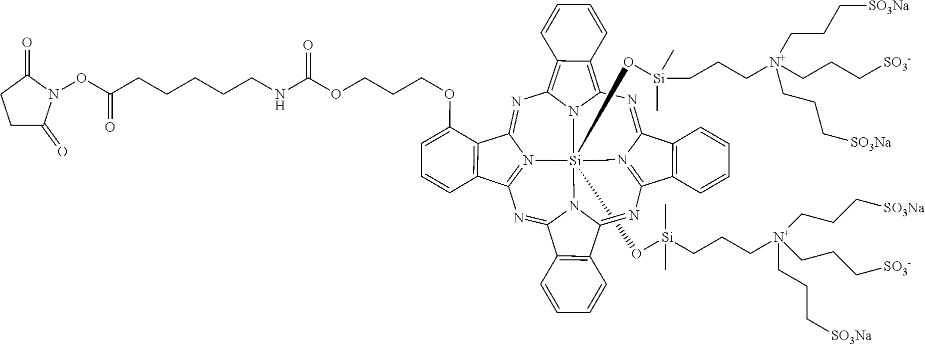

IRDYE 700DX is commercially available from LI-COR (Lincoln, Nebr.). Other variations of IRDye 700DX, which can also be used in the methods and compositions described herein, including the carboxylate derivative, are disclosed in U.S. Pat. No. 7,005,518 (incorporated herein by reference). For additional information see US20150374819A1, and for other examples of cyanine dyes such as IRDye® 800CW see, e.g., U.S. Pat. Nos. 6,995,274; 7,504,089; 7,597,878; 8,227,621; and 8,303,936.

Any method known in the art can be used to covalently link the anti-T cell antibody (or antigen-binding fragment thereof) and the NIR-photoactivated cytotoxin. See, e.g., US20150374819, 6,995,274; 7,005,518; 7,504,089; 7,597,878; 8,227,621; and 8,303,936.

Pharmaceutical Compositions and Methods of Administration

The methods described herein include the use of pharmaceutical compositions comprising the T cell targeting constructs as an active ingredient.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical compositions can be included in a container, pack, or dispenser together with instructions for administration.

Methods of Treatment

The present methods can be used to treat a number of inflammatory diseases associated with the presence of memory T cells, e.g., skin inflammatory diseases such as psoriasis, cutaneous T cell lymphomas, vitiligo, atopic dermatitis, graft vs. host disease, and contact dermatitis; Rheumatoid arthritis; or other inflammatory diseases. Systemic or local administration of a T cell targeting construct as described herein plus administration of NIR light therapy over the chest (e.g., to treat asthma), back (e.g., to treat lupus nephritis) or via endoscope (e.g., to treat inflammatory bowel disease or Crohn's disease) to safely deplete inflammatory T cells.

Generally, the methods include administering a therapeutically effective amount of administering at least one dose of a T cell targeting construct as described herein, and then administering near infrared light therapy to the affected area as described herein, to a subject who is in need of, or who has been determined to be in need of, such treatment. Methods for identifying such subjects are known in the art.

As used in this context, to “treat” means to ameliorate at least one symptom of the disorder associated with presence of memory T cells. For example, a treatment can result in a reduction in inflammation, and/or decreased levels of memory T cells in the tissue.

The methods can include administering NIR light with a wavelength of 660 to 740 nm, e.g., 660 nm, 670 nm, 680 nm, 690 nm, 700 nm, 710 nm, 720 nm, 730 nm, or 740 nm, and can be administered using any method known in the art, e.g., therapeutic laser.

Dosage

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### Examples

The invention is further described in the following examples, which do not limit the scope of the invention described in the claims.

### Example 1. IR700-Conjugated Antibodies+NIR Light Depletes Human T Cells and Specific T Cell Subsets In Vitro

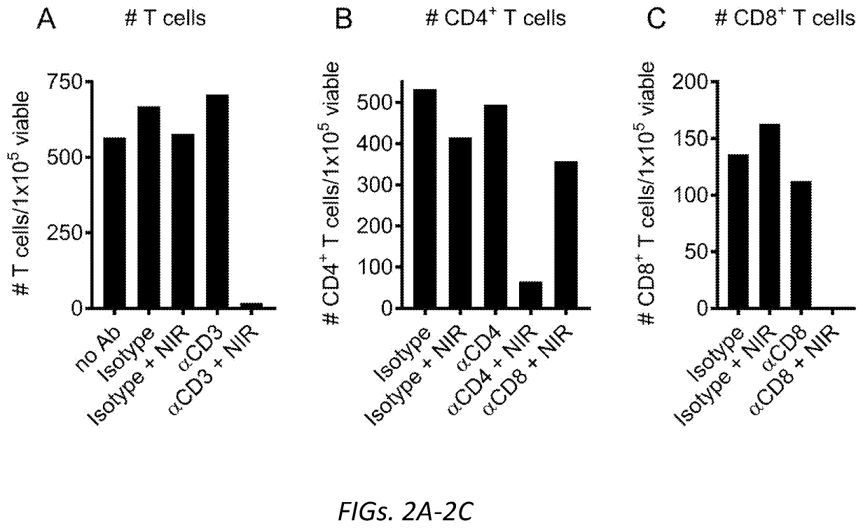

250,000 peripheral blood mononuclear cells isolated from the blood of healthy human donors were cultured in 250 ul Isocoves medium in wells of 48 well plates. Cells were incubated with 12.5 ug of αCD3 or isotype control IR700DX-conjugated antibody for 15 min. Wells that received NIR were treated with 100 J/cm² 690 nm light from high powered LED, incubated for 1 hour, and then immunostained and analyzed by flow cytometry. As shown in FIG. 2A, treatment of cells with both anti-CD3-IR700 and near infrared light (αCD3+NIR) led to near total depletion of CD3+T cells. However, treatment with anti-CD3-IR700 (αCD3) or near infrared light (NIR) alone did not deplete T cells. This data shows that both the antibody and NIR are needed for cell depletion, supporting the idea that, in vivo after systemic treatment with IR700 conjugated antibodies, T cells will be only be depleted in tissues treated with NIR light. Treatment with NIR and an IR700 conjugated isotype control antibody also did not deplete T cells, demonstrating that only cells expressing the antibody targeted antigen are depleted by this method. As shown in FIG. 2B, CD4+ T cells were selectively depleted by anti-CD4-IR700 plus near infrared light (αCD4+NIR), but CD8+ T cells, which lack CD4, were spared. This data demonstrates that this approach can selectively target and destroy specific T cell subsets. As shown in FIG. 2C, CD8+ T cells were selectively depleted by anti-CD8-IR700 plus near infrared light (αCD8+NIR).

### Example 2. In Vivo Depletion of Human T Cells from Human Skin Grafts Using Human Engrafted Mice

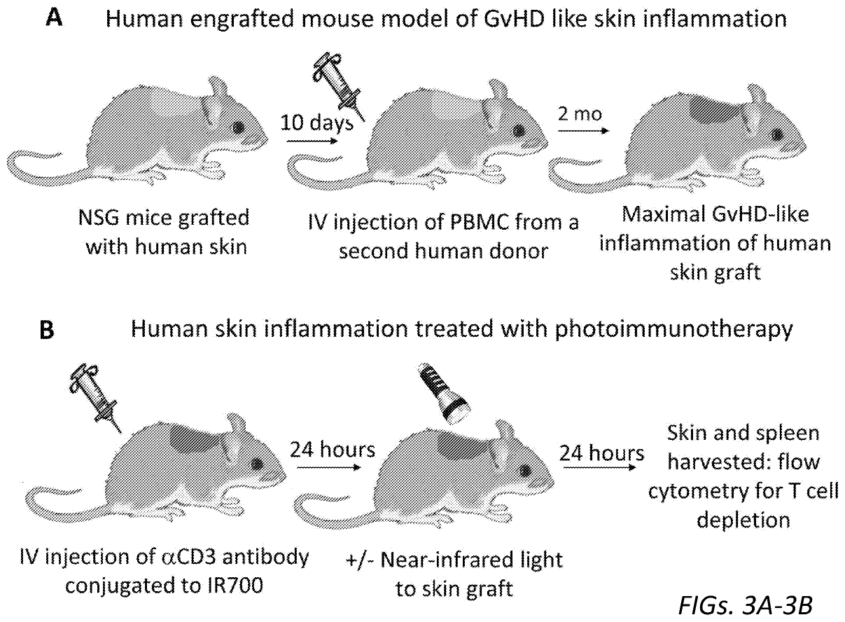

As shown in FIGS. 3A-3B, NSG mice were grafted with adult human skin and subsequently infused IV with PBMC from a second human donor develop a GvHD-like dermatitis within the human skin graft. Data was generated by infusing mice IV with an anti-CD3 antibody conjugated to IR700 and exposing the skin graft to near-infrared light. T cells were then isolated from the skin graft and spleen and analyzed by flow cytometry.

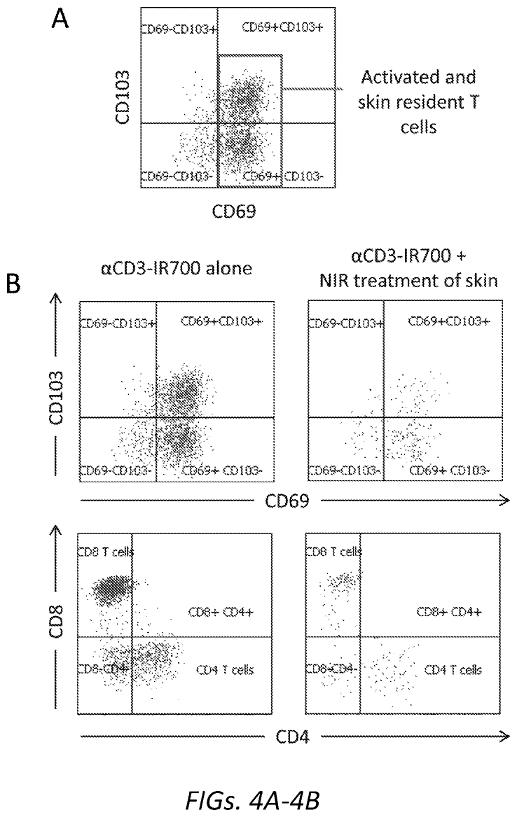

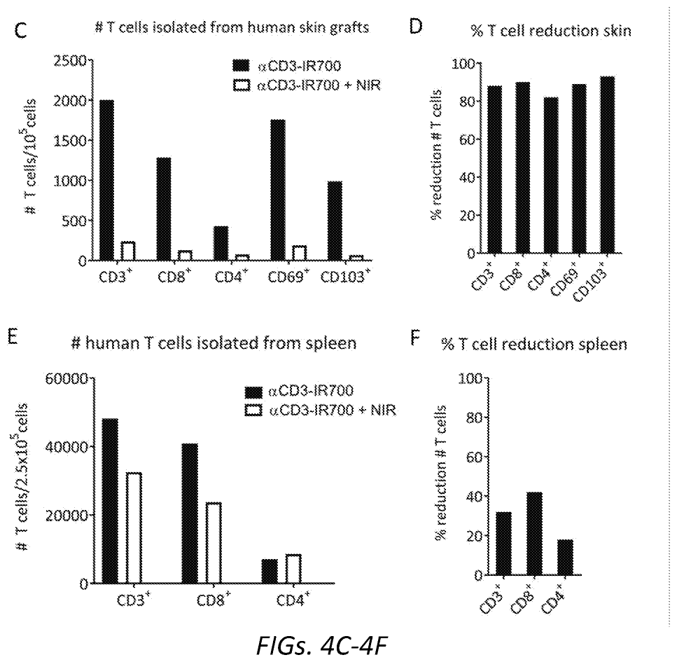

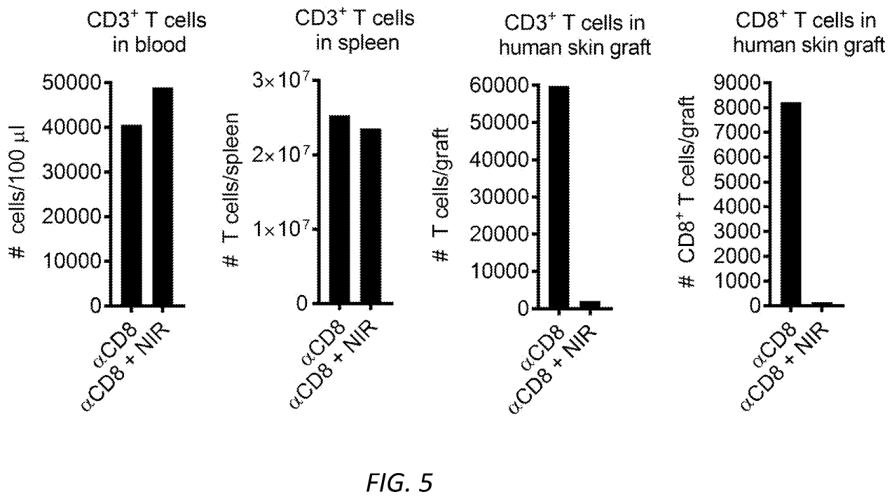

Activated and resident memory T cell were effectively depleted from skin by photoimmunotherapy. Inflamed skin contains activated and resident memory T cells that express CD69 (activated, resident memory) and CD103 (resident memory); T cells isolated from the skin of human engrafted mice are shown in FIG. 4A. These activated and resident T cells were effectively depleted from skin by photoimmunotherapy; compare T cells isolated from the skin grafts of human engrafted mice treated with the antibody conjugate alone (FIG. 4B, left panels) vs. antibody conjugate with near-infrared treatment of the skin graft (FIG. 4B, right panels). Depletion of T cells from the skin and spleen was also seen (see FIGS. 4C and 4E, respectively). 88% of total T cells, 89% of activated and 93% of resident memory T cells were depleted from skin; but spleen total T cells were reduced by only 32% (see FIGS. 4D and 4F, respectively). These data show that photoimmunotherapy specifically and effectively depletes T cells from NIR treated skin.

In addition, selective depletion of T cells in NIR treated skin was achieved in vivo using a second T cell targeting antibody following treatment with anti-CD8-IR700 alone (αCD8) or with a combination of anti-CD8-IR700 and near infrared light delivered to the human skin graft (αCD8+NIR). As shown in FIG. 5, CD8 T cells were depleted selectively in NIR treated skin but were not depleted in the spleen or blood. The few remaining CD3+ T cells in the skin grafts of αCD8+NIR treated mice were CD4+ T cells which were not targeted by the antibody. This data shows the selective depletion of targeted T cells only in peripheral tissues treated with NIR.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A Memory T cell targeting construct comprising an anti-T cell antibody or antigen-binding portion thereof that binds to an antigen present on the surface of a memory T cell, linked to a near-infrared photoactivated cytotoxin.

2. The T cell targeting construct of claim 1, wherein the anti-T cell antibody or antigen-binding portion thereof that binds to CD3, CD4, CD8, CD103, C-X-C motif chemokine receptor 6 (CXCR6), or CD69.

3. The T cell targeting construct of claim 1, wherein the near-infrared photoactivated cytotoxin comprises a phthalocyanine dye.

4. The T cell targeting construct of claim 3, wherein the phthalocyanine dye is IRDYE 700DX.

5. The T cell targeting construct of claim 1, wherein the near-infrared photoactivated cytotoxin comprises a light-activated Platinum IV (PtIV) complex or photoactivatable Re(I) complex and lanthanide-doped upconversion nanoparticles (UCNPs).

6. The T cell targeting construct of claim 5, wherein the light-activated PtIV complex comprises trans, trans, trans-P[(N₃)₂(OH)₂(NH₃)(py)].

7. The T cell targeting construct of claim 1, wherein the antibody is a deimmunised, humanized or fully human antibody.

8. A method of reducing inflammation in a tissue in a subject, the method comprising:
(i) administering to the subject an effective amount of a T cell Targeting Construct of claim 1, and
(ii) administering to the tissue a near infrared (NIR) light sufficient to activate the cytotoxin.

9. The method of claim 8, wherein the NIR light is a wavelength of 660 to 740 nm.

10. The method of claim 9, wherein the T cell Targeting Construct is administered cutaneously or parenterally.

11. The method of claim 10, wherein the parenteral administration is intravenous, intraperitoneal, subcutaneous, or intramuscular.

12. The method of claim 8, wherein the subject has an inflammatory or autoimmune condition.

13. The method of claim 12, wherein the subject has skin lesions associated with psoriasis, atopic dermatitis, lupus, vitiligo, graft-versus-host disease, cutaneous T-cell lymphoma, contact dermatitis, cutaneous hypersensitivity response, lichen planus, lichen planopilaris, rejection of vascularized composite allografts, alopecia areata, scarring alopecia or sarcoid and the methods include administering the NIR light to the skin lesions.

14. The method of claim 12, wherein the subject has T cell mediated kidney or renal pathology/disease associated with lupus nephritis, autoimmune nephritis or kidney graft rejection and the methods include administering the NIR light to the kidney.

15. The method of claim 12, wherein subject has gastrointestinal inflammation as a result of an autoimmune and/or an inflammatory condition, and the methods include administering the NIR light to the gut or bowel of the subject.

16. The method of claim 12, wherein the subject has joint inflammation from rheumatoid arthritis or spondyloarthritides, and the methods include administering the NIR light to affected joints.

17. The method of claim 15, wherein the autoimmune and/or an inflammatory condition is an inflammatory bowel disease.

18. The method of claim 17, wherein the inflammatory bowel disease is Crohn's disease or ulcerative colitis.
